(19) United States
(12) Patent Application Publication
(10) Pub. No.: US 2023/0062390 A1
(43) Pub. Date: Mar. 2, 2023

(54) LIGHT THERAPY APPARATUS AND METHOD FOR FABRICATING THE SAME

(71) Applicant: Samsung Display Co., LTD., Yongin-si (KR)

(72) Inventors: Seong Won KIM, Asan-si (KR); Byeong Hee WON, Incheon (KR); Dong Chul SHIN, Hwaseong-si (KR); Jang Yeol YOON, Seoul (KR)

(21) Appl. No.: 17/898,430

(22) Filed: Aug. 29, 2022

(30) Foreign Application Priority Data
Aug. 30, 2021 (KR) 10-2021-0114695

(51) Int. Cl. H01L 27/32; H01L 51/50; H01L 51/00; H01L 51/56; A61N 5/06

(52) U.S. Cl. CPC H01L 27/3206; H01L 51/5088; H01L 51/0097; H01L 51/56; A61N 5/06; H01L 2251/5338; A61N 2005/0652; A61N 2005/0653; A61N 2005/0632

(57) ABSTRACT

A light therapy apparatus includes a first source voltage line including a first sub-source voltage line and a second sub-source voltage line, which are electrically insulated from each other, a first light emitting element connected to the first sub-source voltage line, where the first light emitting element includes a first intermediate layer, and a second light emitting element connected to the second sub-source voltage line, where the second light emitting element includes a second intermediate layer. The first intermediate layer has a first thickness, and the second intermediate layer has a second thickness greater than the first thickness.

This application claims priority to Korean Patent Application No. 10-2021-0114695, filed on Aug. 30, 2021, and all the benefits accruing therefrom under 35 U.S.C. § 119, the content of which in its entirety is herein incorporated by reference.

### BACKGROUND

### 1. Field

Embodiments of the invention relate to a light therapy apparatus and a method for fabricating the light therapy apparatus.

### 2. Description of the Related Art

Currently, in medical fields, light therapy has attracted attention as a means of health improvement or therapy. The light therapy is a technology of irradiating a therapy target site with light of a specific wavelength having a therapeutic effect for a predetermined time, or is a technique of activating, regenerating, or destroying a specific tissue in the skin of a human body by absorbing the light into the skin. Additionally, the light used for light therapy promotes biochemical reactions of skin cells, and may thus be applied to various fields such as wound treatment, acne, psoriasis, whitening, and wrinkle treatment.

A light therapy apparatus may use an organic light emitting diode (“OLED”). The OLED may emit light by recombination of electrons and holes.

### SUMMARY

In a light therapy apparatus, it is desired to improve portability thereof to allow a user to do another activity while using the light therapy apparatus and it is desired to improve accessibility thereof to allow the user to use the light therapy apparatus without going to the hospital or the like.

Embodiments of the invention provide a light therapy apparatus of which a cost is decreased and power used for driving is decreased, and a method for fabricating the light therapy apparatus.

An embodiment of the invention provides a light therapy apparatus including a first source voltage line including a first sub-source voltage line and a second sub-source voltage line, which are electrically insulated from each other, a first light emitting element connected to the first sub-source voltage line, where the first light emitting element includes a first intermediate layer, and a second light emitting element connected to the second sub-source voltage line, wherein the second light emitting element includes a second intermediate layer. In such an embodiment, the first intermediate layer has a first thickness, and the second intermediate layer has a second thickness greater than the first thickness.

In an embodiment, each of the first intermediate layer and the second intermediate layer may include a first hole injection layer, a hole transport layer, an emission layer, an electron transport layer, and an electron injection layer, which are sequentially stacked one on another. In such an embodiment, the second intermediate layer may further include a second hole injection layer.

In an embodiment, the second hole injection layer of the second intermediate layer may be disposed between the first hole injection layer and the hole transport layer of the second intermediate layer.

In an embodiment, a wavelength of light emitted from the first light emitting element and a wavelength of light emitted from the second light emitting element may be different from each other.

In an embodiment, the light therapy apparatus may further include a substrate including a plurality of islands divided by a plurality of cutout portions and a plurality of bridges, each connecting adjacent islands among the plurality of islands to each other. In such an embodiment, the first source voltage line, the first light emitting element, and the second light emitting element may be disposed on the substrate.

In an embodiment, both of the first light emitting element and the second light emitting element may be disposed on each of the plurality of islands.

In an embodiment, one of the first light emitting element and the second light emitting element may be disposed on each of the plurality of islands.

In an embodiment, the first light emitting elements may be arranged along a first direction, the second light emitting elements are arranged along the first direction, and the first light emitting elements and the second light emitting elements may be alternately disposed along a second direction crossing the first direction.

An embodiment of the invention provides a light therapy apparatus including a substrate including a plurality of islands divided by a plurality of cutout portions and a plurality of bridges, each connecting adjacent islands among the plurality of islands to each other, a first conductive layer disposed on the substrate, where the first conductive layer includes a first sub-source voltage line and a second sub-source voltage line, which are electrically insulated from each other, a first planarization layer disposed on the first conductive layer, a second conductive layer disposed on the first planarization layer, where the second conductive layer includes a plurality of first electrodes, a bank layer disposed on the second conductive layer, where the bank layer exposes each of the plurality of first electrodes, and an intermediate layer including a first intermediate layer disposed on a first sub-electrode of the plurality of first electrodes exposed by the bank layer and a second intermediate layer disposed on a second sub-electrode of the plurality of first electrodes exposed by the bank layer. In such an embodiment, the first sub-source voltage line is electrically connected to the first sub-electrode, and the second sub-source voltage line is electrically connected to the second sub-electrode.

In an embodiment, the first intermediate layer may have a first thickness, and the second intermediate layer has a second thickness greater than the first thickness.

In an embodiment, each of the first intermediate layer and the second intermediate layer may include a first hole injection layer, a hole transport layer, an emission layer, an electron transport layer, and an electron injection layer that are sequentially stacked. The second intermediate layer may further include a second hole injection layer.

In an embodiment, the second hole injection layer of the second intermediate layer may be disposed between the first hole injection layer and the hole transport layer of the second intermediate layer.

In an embodiment, one of the first sub-electrode and the second sub-electrode may be disposed on each of the plurality of islands.

In an embodiment, the first sub-electrode and the second sub-electrode may be arranged along a first direction, respectively, and the first sub-electrode and the second sub-electrode may be alternately disposed along a second direction crossing the first direction.

In an embodiment, the first sub-electrode and the second sub-electrode may be disposed on different islands among the plurality of islands, respectively.

In an embodiment, the light therapy apparatus may further include an emission area defined by the bank layer, where the emission area may emit light. In such an embodiment, the emission area may include a first emission area overlapping the first sub-electrode and a second emission area overlapping the second sub-electrode.

an embodiment, the light therapy apparatus may further include a cathode electrode disposed on the intermediate layer.

An embodiment of the invention provides method for fabricating a light therapy apparatus including providing a first conductive layer including a first sub-electrode and a second sub-electrode on a substrate, providing a bank layer on the first conductive layer to expose each of the first sub-electrode and the second sub-electrode, and providing a first hole injection layer on the first sub-electrode and the second sub-electrode exposed by the bank layer, and providing a second hole injection layer disposed on the first hole injection layer on the second sub-electrode. In such an embodiment, the providing the first hole injection layer is performed through a first deposition mask, and the providing the second hole injection layer is performed through a second deposition mask different from the first deposition mask. In such an embodiment, the first deposition mask exposes the first sub-electrode and the second sub-electrode exposed by the bank layer, and the second deposition mask covers the first sub-electrode exposed by the bank layer, and exposes the second sub-electrode exposed by the bank layer.

In an embodiment, the first conductive layer, which is disposed on the substrate, may further include a first sub-source voltage line and a second sub-source voltage line electrically, which are insulated from each other. In such an embodiment, the first sub-source voltage line may be electrically connected to the first sub-electrode, and the second sub-source voltage line may be electrically connected to the second sub-electrode.

In an embodiment, the substrate may include a plurality of islands divided by a plurality of cutout portions and a plurality of bridges, each connecting adjacent islands among the plurality of islands to each other.

In embodiments according to the invention, a cost of the light therapy apparatus may be decreased, and power used for driving of the light therapy apparatus may be decreased, such that portability or the like of the light therapy apparatus may be improved.

### BRIEF DESCRIPTION OF THE DRAWINGS

These and/or other features of the invention will become apparent and more readily appreciated from the following description of the embodiments, taken in conjunction with the accompanying drawings, in which:

FIG. 1 is a perspective view of a light therapy apparatus according to an embodiment;

FIG. 2 is a perspective view illustrating an embodiment of the light therapy apparatus of FIG. 1 in a state in which the light therapy apparatus is stretched in a horizontal direction;

FIG. 3 is a perspective view illustrating a an embodiment of the light therapy apparatus of FIG. 1 in a state in which the light therapy apparatus is locally stretched;

FIG. 4 is a schematic plan view illustrating a first source voltage line and a second source voltage line of the light therapy apparatus according to an embodiment;

FIG. 5 is a circuit diagram of one light emitting element of the light therapy apparatus according to an embodiment;

FIG. 6 is a schematic plan view illustrating islands of a substrate in a partial area of the light therapy apparatus according to an embodiment;

FIG. 7 is a plan view of an array repeating unit according to an embodiment;

FIG. 8 is a cross-sectional view taken along line VIII-VIII′ of FIG. 7;

FIG. 9 is an enlarged view of the periphery of an organic layer of FIG. 8;

FIGS. 10 to 12 are cross-sectional views for describing a method for fabricating the light therapy apparatus according to an embodiment;

FIG. 13 is a plan view of a partial area of a light therapy apparatus according to an alternative embodiment; and

FIG. 14 is a plan view of a partial area of a light therapy apparatus according to another alternative embodiment.

### DETAILED DESCRIPTION

Embodiments of the invention will now be described more fully hereinafter with reference to the accompanying drawings, in which preferred embodiments of the invention are shown. The invention may, however, be embodied in different forms and should not be construed as limited to the embodiments set forth herein. Rather, these embodiments are provided so that this disclosure will be thorough and complete, and will fully convey the scope of the invention to those skilled in the art. The same reference numbers indicate the same components throughout the specification. In the attached drawing figures, the thickness of layers and regions is exaggerated for clarity.

It will be understood that, although the terms “first,” “second,” “third” etc. may be used herein to describe various elements, components, regions, layers and/or sections, these elements, components, regions, layers and/or sections should not be limited by these terms. These terms are only used to distinguish one element, component, region, layer or section from another element, component, region, layer or section. Thus, “a first element,” “component,” “region,” “layer” or “section” discussed below could be termed a second element, component, region, layer or section without departing from the teachings herein.

It will also be understood that when a layer is referred to as being “on” another layer or substrate, it can be directly on the other layer or substrate, or intervening layers may also be present. In contrast, when an element is referred to as being “directly on” another element, there are no intervening elements present.

The terminology used herein is for the purpose of describing particular embodiments only and is not intended to be limiting. As used herein, the singular forms “a,” “an,” and “the” are intended to include the plural forms, including “at least one,” unless the content clearly indicates otherwise. “Or” means “and/or.” “At least one of A and B” means “A and/or B.” As used herein, the term “and/or” includes any and all combinations of one or more of the associated listed items. It will be further understood that the terms “comprises” and/or “comprising,” or “includes” and/or “including” when used in this specification, specify the presence of stated features, regions, integers, steps, operations, elements, and/or components, but do not preclude the presence or addition of one or more other features, regions, integers, steps, operations, elements, components, and/or groups thereof.

“About” or “approximately” as used herein is inclusive of the stated value and means within an acceptable range of deviation for the particular value as determined by one of ordinary skill in the art, considering the measurement in question and the error associated with measurement of the particular quantity (i.e., the limitations of the measurement system).

Unless otherwise defined, all terms (including technical and scientific terms) used herein have the same meaning as commonly understood by one of ordinary skill in the art to which this disclosure belongs. It will be further understood that terms, such as those defined in commonly used dictionaries, should be interpreted as having a meaning that is consistent with their meaning in the context of the relevant art and the disclosure, and will not be interpreted in an idealized or overly formal sense unless expressly so defined herein.

Embodiments are described herein with reference to cross section illustrations that are schematic illustrations of idealized embodiments. As such, variations from the shapes of the illustrations as a result, for example, of manufacturing techniques and/or tolerances, are to be expected. Thus, embodiments described herein should not be construed as limited to the particular shapes of regions as illustrated herein but are to include deviations in shapes that result, for example, from manufacturing. A region illustrated or described as flat may, typically, have rough and/or nonlinear features, for example. Moreover, sharp angles that are illustrated may be rounded. Thus, the regions illustrated in the drawing figures are schematic in nature and their shapes are not intended to illustrate the precise shape of a region and are not intended to limit the scope of the claims.

Hereinafter, embodiments of the invention will be described in detail with reference to the accompanying drawings.

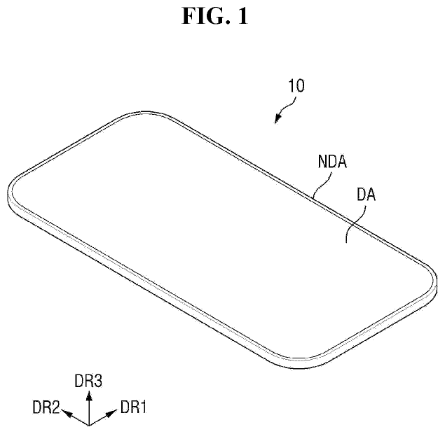

FIG. 1 is a perspective view of a light therapy apparatus according to an embodiment.

Throughout the specification, a first direction DR1 and a second direction DR2 are different directions, and cross each other. It is illustrated in the drawings that the first direction DR1 indicates a transverse direction in a plan view and the second direction DR2 indicates a longitudinal direction in a plan view, but the present disclosure is not limited thereto. In the following embodiments, one side in the first direction DR1 refers to a right direction in a plan view, the other side in the first direction DR1 refers to a left direction in a plan view, one side in the second direction DR2 refers to an upper direction in a plan view, and the other side in the second direction DR2 refers to a lower direction in a plan view.

A third direction DR3 is a direction crossing a plane on which the first direction DR1 and the second direction DR2 are placed, and perpendicularly crosses both the first direction DR1 and the second direction DR2. However, it will be understood that directions mentioned in embodiments are relative directions, and embodiments are not limited to the mentioned directions.

Unless otherwise defined, in the present specification, the terms “upper”, “upper surface”, and “upper side” expressed with respect to the third direction DR3 refer to a light emitting surface side with respect to the light therapy apparatus 10, and the terms “lower”, “lower surface”, and “lower side” refer to an opposite side to a light emitting surface with respect to the light therapy apparatus 10.

Referring to FIG. 1, an embodiment of the light therapy apparatus 10 is an apparatus that emits light of a specific wavelength having a therapeutic effect. An element emitting light in the light therapy apparatus 10 may include an organic light emitting diode (“OLED”), but is not limited thereto. Hereinafter, embodiments where the light emitting element is an OLED will be described in detail, but another type of light emitting element known in the art may be applied as the light emitting element without departing from the spirit and scope of the disclosure.

The light therapy apparatus 10 may further include a touch member, a sensor, various controllers, a housing, and other components in addition to the light emitting elements. It will be understood that any apparatus that emits light having a therapeutic effect corresponds to the light therapy apparatus 10 regardless of a main use, an added function, a name, or the like, thereof. In an embodiment, the light therapy apparatus 10 may activate, regenerate, or destroy a specific tissue in the skin of a human body by irradiating a therapy target site with light for a predetermined time to absorb the light into the skin. In an embodiment, the light used for light therapy promotes biochemical reactions of skin cells, and may thus be applied to various fields such as wound treatment, acne, psoriasis, whitening, and wrinkle treatment.

The light therapy apparatus 10 may include a therapy area DA and a non-therapy area NDA. The therapy area DA is an area in which therapy is performed by emitting light and may be an active area, and the non-therapy area NDA is an area in which light is not emitted and therapy is not performed and may be an inactive area. The therapy area DA may have a rectangular shape in a plan view, but is not limited thereto, and alternatively, the therapy area DA may have one of other various shapes such as a square shape, a rhombic shape, a circular shape, and an elliptical shape in a plan view.

The non-therapy area NDA may be disposed around the therapy area DA. The non-therapy area NDA may completely or partially surround the therapy area DA. Signal lines for applying signals to the therapy area DA or transmitting signals detected in the therapy area DA may be disposed in the non-therapy area NDA.

The light therapy apparatus 10 may be a flexible apparatus. The light therapy apparatus 10 may be stretched, curved, bent, folded, or rolled. Flexibility of such a light therapy apparatus 10 may be implemented through a flexible substrate. The flexible substrate may include a flexible polymer. In an embodiment, for example, the flexible polymer may include a polymer such as polyimide or polyester (e.g., polyethylene terephthalate, polybutylene phthalate, polyethylene naphthalate, etc.), polystyrene, polycarbonate, polyether sulfone, polyarylate, polycycloolefin, norbornen resin, poly(chlorotrifluoroethylene), or polymethyl methacrylate.

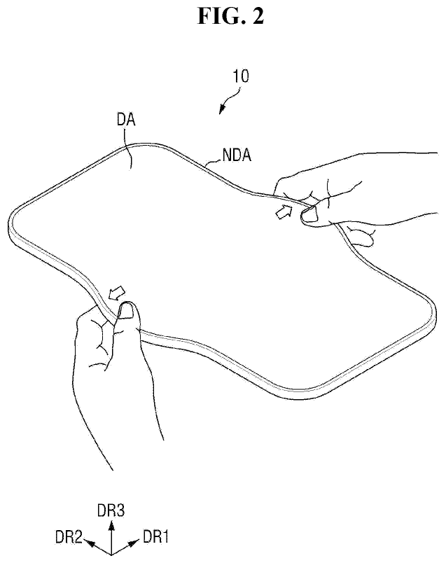

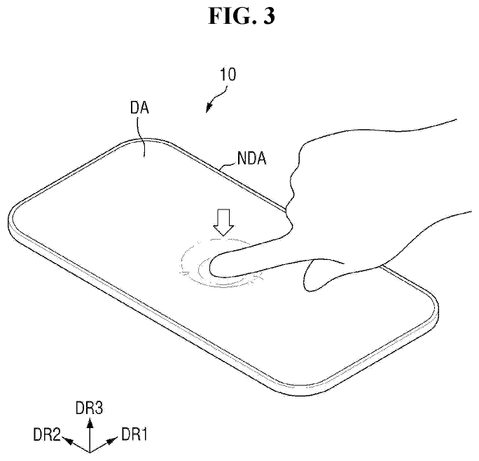

In FIGS. 2 and 3, an embodiment of a flexible light therapy apparatus, where the flexible light therapy apparatus is a flexible light therapy apparatus, is illustrated. FIG. 2 is a perspective view illustrating an embodiment of the light therapy apparatus of FIG. 1 in a state in which the light therapy apparatus is stretched in a horizontal direction. FIG. 3 is a perspective view illustrating an embodiment of the light therapy apparatus of FIG. 1 in a state in which the light therapy apparatus is locally stretched.

Referring to FIG. 2, an embodiment of the light therapy apparatus 10 may be stretched in the horizontal direction. In an embodiment, for example, when a user grasps the vicinities of edges of the light therapy apparatus 10 and stretches the light therapy apparatus 10 toward opposing sides, the light therapy apparatus 10 may be stretched toward the opposing sides. An area of the light therapy apparatus 10 in a plan view may be increased due to the stretching. In an embodiment, the light therapy apparatus 10 may be stretched in the first direction DR1 as shown in FIG. 2, the light therapy apparatus 10 may also be stretched in the second direction DR2, stretched in both the second direction DR2 and the first direction DR1, or stretched in another horizontal direction. The light therapy apparatus 10 may be stretched by an external force, and may contract and return to its original state when the external force is removed.

Referring to FIG. 3, an embodiment of the light therapy apparatus 10 may be locally stretched while maintaining an overall area in a plan view. In such an embodiment, as illustrated in FIG. 3, when the light therapy apparatus 10 is pressed in the third direction DR3, which is a thickness direction, the light therapy apparatus 10 may be locally stretched around a pressed point. In this case, a direction in which the light therapy apparatus 10 is stretched may be a direction inclined with respect to the horizontal direction, and an overall area of the light therapy apparatus 10 in a plan view may be maintained as a same area as an area of the light therapy apparatus 10 before the light therapy apparatus 10 is pressed. When a pressing force is removed, a stretched portion of the light therapy apparatus 10 may contract again and return to its original state.

In an embodiment, the stretching of FIG. 2 and the stretching of FIG. 3 may be performed simultaneously. In an embodiment, for example, an area of the light therapy apparatus 10 in a plan view may be further increased as a whole while the light therapy apparatus 10 is locally stretched in the direction inclined with respect to the horizontal direction with respect to the pressing in the thickness direction.

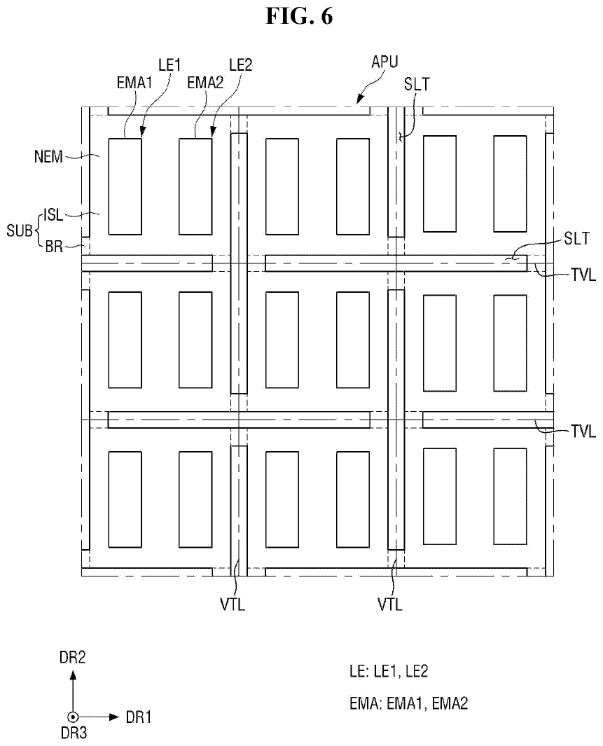

When the light therapy apparatus 10 is stretched or contracted as described above, stress is applied to a substrate of the light therapy apparatus 10 and thin films disposed on the substrate. In an embodiment, a substrate SUB (see FIG. 6) of the light therapy apparatus 10 may include a cutout portion SLT (see FIG. 6) to alleviate such stretching and contracting stress. A detailed description therefor will be provided later.

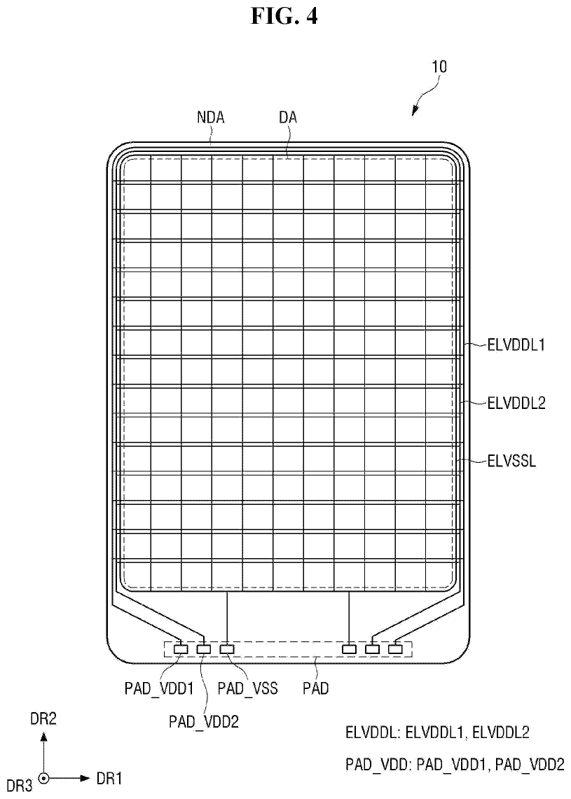

FIG. 4 is a schematic view illustrating a layout of a first source voltage line and a second source voltage line of the light therapy apparatus according to an embodiment in a plan view.

Referring to FIG. 4, an embodiment of the light therapy apparatus 10 may include a first source voltage line ELVDDL, a second source voltage line ELVSSL, and a pad part PAD.

The first source voltage line ELVDDL may include a first sub-source voltage line ELVDDL1 and a second sub-source voltage line ELVDDL2. The first sub-source voltage line ELVDDL1 and the second sub-source voltage line ELVDDL2 may be separated from each other. The first sub-source voltage line ELVDDL1 and the second sub-source voltage line ELVDDL2 may be electrically separated or insulated from each other. As described later, accordingly, light emitting elements connected to the first sub-source voltage line ELVDDL1 and light emitting elements connected to the second sub-source voltage line ELVDDL2 may be driven independently of each other.

The pad part PAD may include a first source voltage pad part PAD_VDD electrically connected to the first source voltage line ELVDDL and a second source voltage pad part PAD_VSS electrically connected to the second source voltage line ELVSSL. The first source voltage pad part PAD_VDD may include a first sub-source voltage pad part PAD_VDD1 electrically connected to the first sub-source voltage line ELVDDL1 and a second sub-source voltage pad part PAD_VDD2 electrically connected to the second sub-source voltage line ELVDDL2. The pad part PAD may be disposed on a side of the therapy area DA in the second direction DR2, but is not limited thereto.

The first sub-source voltage line ELVDDL1 may be disposed to traverse the therapy area DA in the first direction DR1. In an embodiment, at least a portion of the first sub-source voltage line ELVDDL1 may be disposed in the non-therapy area NDA. The first sub-source voltage line ELVDDL1 disposed in the non-therapy area NDA may extend from the pad part PAD to one side in the second direction DR2, bypass the therapy area DA, and extend to the pad part PAD again.

At least a portion of the first sub-source voltage line ELVDDL1 may traverse the therapy area DA in the first direction DR1. The first sub-source voltage line ELVDDL1 traversing the therapy area DA may extend from the first sub-source voltage line ELVDDL1 disposed in the non-therapy area NDA positioned on one side of the therapy area DA in the first direction DR1 to the first sub-source voltage line ELVDDL1 disposed in the non-therapy area NDA positioned on an opposing side of the therapy area DA in the first direction DR1.

A layout of the second sub-source voltage line ELVDDL2 in a plan view may be substantially the same as that of the first sub-source voltage line ELVDDL1 in a plan view, but is not limited thereto. The first sub-source voltage line ELVDDL1 and the second sub-source voltage line ELVDDL2 may not overlap each other except for a crossing portion therebetween. In such an embodiment, the first sub-source voltage line ELVDDL1 and the second sub-source voltage line ELVDDL2 may be disposed adjacent to each other in a plan view.

The second source voltage line ELVSSL may be disposed to traverse the therapy area DA in the second direction DR2. In an embodiment, at least a portion of the second source voltage line ELVSSL may be disposed in the non-therapy area NDA. The second source voltage line ELVSSL disposed in the non-therapy area NDA may extend from the pad part PAD to one side in the second direction DR2, bypass the therapy area DA, and extend to the pad part PAD again. The second source voltage line ELVSSL may surround the therapy area DA in a plan view, but is not limited thereto.

At least a portion of the second source voltage line ELVSSL may traverse the therapy area DA in the second direction DR2. The second source voltage line ELVSSL traversing the therapy area DA may extend from a second source voltage line ELVSSL disposed in the non-therapy area NDA positioned on one side of the therapy area DA in the second direction DR2 to a second source voltage line ELVSSL disposed in the non-therapy area NDA positioned on an opposing side of the therapy area DA in the second direction DR2.

In the therapy area DA, the first sub-source voltage line ELVDDL1 and the second sub-source voltage line ELVDDL2 may extend substantially in the first direction DR1 and the second source voltage line ELVSSL may extend substantially in the second direction DR2, but the first sub-source voltage line ELVDDL1, the second sub-source voltage line ELVDDL2, and the second source voltage line ELVSSL may include bent portions in the therapy area DA. The first sub-source voltage line ELVDDL1, the second sub-source voltage line ELVDDL2, and the second source voltage line ELVSSL may be bent in the therapy area DA to bypass a substrate SUB (see FIG. 6) and a cutout portion SLT (see FIG. 6) of the light therapy apparatus 10.

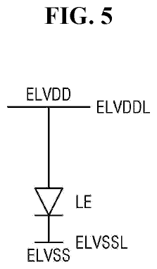

FIG. 5 is a circuit diagram of one light emitting element of the light therapy apparatus according to an embodiment.

Referring to FIG. 5, in an embodiment, the light emitting element LE may include a first electrode (or an anode electrode), a second electrode (or a cathode electrode), and an intermediate layer (or an organic layer) interposed between the first electrode and the second electrode. The first electrode of the light emitting element LE may be electrically connected to the first source voltage line ELVDDL for applying a first source voltage ELVDD, and the second electrode of the light emitting element LE may be electrically connected to the second source voltage line ELVSSL for applying a second source voltage ELVSS.

However, the disclosure is not limited thereto, and alternatively, the light therapy apparatus 10 may include at least one thin film transistor, a storage capacitor, and the like, electrically connected to the light emitting element LE.

The light emitting element LE may include a first light emitting element LE1 (see FIG. 6) and a second light emitting element LE2 (see FIG. 6) that emit light of different wavelengths, and the first source voltage line ELVDDL may include a first sub-source voltage line ELVDDL1 electrically connected to the first light emitting element LE1 (see FIG. 6) and a second sub-source voltage line ELVDDL2 electrically connected to the second light emitting element LE2 (see FIG. 6). The first sub-source voltage line ELVDDL1 and the second sub-source voltage line ELVDDL2 may be separated and electrically insulated from each other, and accordingly, the first light emitting element LE1 (see FIG. 6) and the second light emitting element LE2 (see FIG. 6) may be driven independently of each other. At least one of the first light emitting element LE1 and the second light emitting element LE2 may be selected and driven based on a therapeutic purpose, such that power consumption of the light therapy apparatus 10 may be decreased.

FIG. 6 is a schematic view illustrating a layout of islands of a substrate in a partial area of the light therapy apparatus according to an embodiment.

Referring to FIG. 6, an embodiment of the light therapy apparatus 10 may include a substrate SUB that supports respective components disposed thereon. In an embodiment, the substrate SUB may have a multilayer structure or a stacked structure, and in such an embodiment, an inorganic film and/or an amorphous silicon layer may be further disposed between respective layers. In an embodiment, for example, the substrate SUB may include a flexible polymer.

The substrate SUB may include a plurality of islands ISL divided by cutout portions SLT and a plurality of bridges BR connected between the islands ISL adjacent to each other. The substrate SUB may have a shape in which an island ISL and bridges BR connected to edges of the island ISL are arranged repeatedly along the first direction DR1 and the second direction DR2.

Each of the plurality of bridges BR may protrude from one island ISL toward an adjacent island ISL in the first direction DR1 or the second direction DR2. The cutout portions SLT may be openings defined through the substrate SUB or formed by removing portions of the substrate SUB. In the cutout portions SLT, the substrate SUB may not be disposed, and separate stacked structures disposed on the substrate SUB may not be disposed. In an embodiment, the cutout portions SLT may be openings formed by removing portions of the substrate SUB and each of the stacked structures disposed on the substrate SUB.

The island ISL and the bridge BR may be integrally formed with each other as a single unitary unit, but are not limited thereto. The islands ISL adjacent to each other may be at least partially connected to each other by the bridge BR, and may be spaced apart from each other and face each other in the remaining portion. The cutout portion SLT may be positioned in an area where the islands ISL adjacent to each other are spaced apart from each other and face each other. In such an embodiment, the islands ISL adjacent to each other may be partially separated from each other by the cutout portion SLT.

Arrangement patterns of the plurality of the islands ISL and bridges BR may be repeatedly arranged based on substantially a same basic unit (hereinafter, referred to as an array repeating unit APU). The array repeating units APU may have substantially the same array of the islands ISL and the bridges BR, and lines, electrodes, or the like, disposed in corresponding areas may also have substantially a same pattern and a same array as each other. The array repeating unit APU may have a square or rectangular shape.

The array repeating units APU may be continuously arranged along the first direction DR1 and second direction DR2, and neighboring array repeating units APU may have a symmetrical relationship with respect to a line perpendicular to an arrangement direction. In an embodiment, for example, the array repeating units APU neighboring to each other in the second direction DR2 may have a line symmetrical shape with respect to a boundary line in the first direction DR1 traversing therebetween (or a boundary therebetween). In such an embodiment, the array repeating units APU neighboring to each other in the first direction DR1 may have a line symmetrical shape with respect to a boundary line in the second direction DR2 traversing therebetween.

The island ISL is positioned at a central portion of the array repeat unit APU. The island ISL may be disposed in one array repeating unit APU, and may be connected to an island ISL of a neighboring array repeating unit APU through the bridge BR. Each of the islands ISL may have a quadrangular shape in a plan view, but is not limited thereto.

The bridges BR may have shapes protruding from the islands ISL to an upper side (one side in the second direction DR2), a lower side (the other side in the second direction DR2), a left side (the other side in the first direction DR1), and a right side (one side in the first direction DR1) in a plan view. All of the respective bridges BR are connected to the islands ISL.

The center of the bridge BR protruding from the island ISL to the left side may be disposed to be biased toward any one of the upper side and the lower side of a center line TVL in the transverse direction, and the center of the bridge BR protruding from the island ISL to the right side may be disposed to be biased toward the other of the upper side and the lower side of the center line TVL in the transverse direction. The bridge BR protruding from the island ISL to the left side and the bridge BR protruding from the island ISL to the right side may have substantially similar areas, and may have a substantially point symmetric relationship with respect to the center of the array repeating unit APU.

The center of the bridge BR protruding from the island ISL to the lower side may be disposed to be biased toward any one of the left side and the right side of an imaginary center line VTL in the longitudinal direction or the second direction DR2, and the center of the bridge BR protruding from the island ISL to the upper side may be disposed to be biased toward the other of the left side and the right side of the imaginary center line VTL in the longitudinal direction. The bridge BR protruding from the island ISL to the lower side and the bridge BR protruding from the island ISL to the upper side may have substantially similar areas, and may have a substantially point symmetric relationship with respect to the center of the array repeating unit APU.

In an embodiment, another island ISL adjacent to the island ISL may be connected to any one of the bridge BR protruding from the island ISL to the left side, the bridge BR protruding from the island ISL to the right side, the bridge BR protruding from the island ISL to the lower side, and the bridge BR protruding from the island ISL to the upper side. The bridge BR connecting between the islands ISL disposed in each of the array repeating units APU adjacent to each other may traverse the array repeating units APU adjacent to each other. In such an embodiment, the islands ISL positioned in each of the array repeating units APU adjacent to each other may be connected to each other through the bridge BR traversing the array repeating units APU adjacent to each other.

The cutout portion SLT is defined through the substrate SUB in the thickness direction. In an embodiment, the substrate SUB may be physically removed in the cutout portion SLT. A material constituting the substrate SUB may not exist in the cutout portion SLT. The cutout portion SLT in which the material constituting the substrate SUB does not exist may be more freely changed in width with respect to stretch and contraction than a portion filled with the material constituting the substrate SUB. Accordingly, when the light therapy apparatus 10 (see FIG. 1) is partially stretched and contracted, the light therapy apparatus 10 is easily stretched and contracted by the cutout portion SLT, such that strain applied to the substrate SUB may be decreased.

In an embodiment, the cutout portion SLT may be defined through not only the substrate SUB but also all of the components, such as an insulating film, disposed on the substrate SUB in the thickness direction. In such an embodiment, the cutout portion SLT may be completely empty as an empty space. However, the disclosure is not limited thereto. In such an embodiment, when the light therapy apparatus 10 (see FIG. 1) is partially stretched and contracted, the light therapy apparatus 10 is easily stretched and contracted by the cutout portion SLT, such that strain applied to the light therapy apparatus 10 (see FIG. 1) may be further decreased. In such an embodiment, in a state in which the light therapy apparatus 10 (see FIG. 1) is attached to the skin, a user may perform a more natural action.

When the substrate SUB is stretched, an interval between the islands ISL may be increased while the bridges BR are stretched. That is, an area of the cutout portion SLT may be increased. In an embodiment, when the substrate SUB is stretched, a shape of each island ISL may not be changed. When the shape of the island ISL is not changed, a width and thickness of the island ISL are not increased or decreased, and thus, a structure of the light emitting element LE formed on the island ISL may not be changed. However, the disclosure is not limited thereto, and alternatively, the shape of each island ISL may also be changed.

The substrate SUB may have a structure in which the entirety thereof is stretchable, but the disclosure is not limited thereto, and alternatively, the substrate SUB may have a structure in which at least a portion thereof is stretchable.

One or more of a plurality of light emitting elements LE may be disposed in the array repeating unit APU. In an embodiment, for example, the plurality of light emitting elements LE may include a first light emitting element LE1 and a second light emitting element LE2 that emit light of different wavelengths from each other. The first light emitting element LE1 and the second light emitting element LE2 may be disposed for each array repeating unit APU, and may be disposed in each island ISL. However, the number of light emitting elements LE disposed in each array repeating unit APU is not limited thereto. The first light emitting element LE1 and the second light emitting element LE2 may be disposed adjacent to each other in the first direction DR1.

Each of the light emitting elements LE in the array repeating unit APU includes a first electrode. The first electrode of the first light emitting element LE1 and the first electrode of the second light emitting element LE2 may be connected to different first source voltage lines, respectively, to receive separate voltages. Each of the first electrodes may be disposed inside the island ISL of the substrate SUB. Each of the first electrodes may not be disposed in the cutout portion SLT of the substrate SUB. In an embodiment, both of the first electrodes of the first light emitting element LE1 and the second light emitting element LE2 disposed in one array repeating unit APU may be disposed on one island ISL.

A plurality of lines are provided to drive each of the first electrodes of the light emitting elements LE. In an embodiment, a plurality of lines, electrodes, insulating films, semiconductor layers, and the like, may be disposed on the substrate SUB. The plurality of lines, electrodes, insulating films, semiconductor layers, and the like, may be disposed not only on the island ISL but also on the bridge BR, but may not be disposed in the cutout portion SLT. However, the disclosure is not limited thereto. In an embodiment, for example, where only the substrate SUB is removed in the cutout portion SLT and insulating films or the like are disposed in the cutout portion SLT, the plurality of lines, electrodes, and the like, may be disposed in the cutout portion SLT.

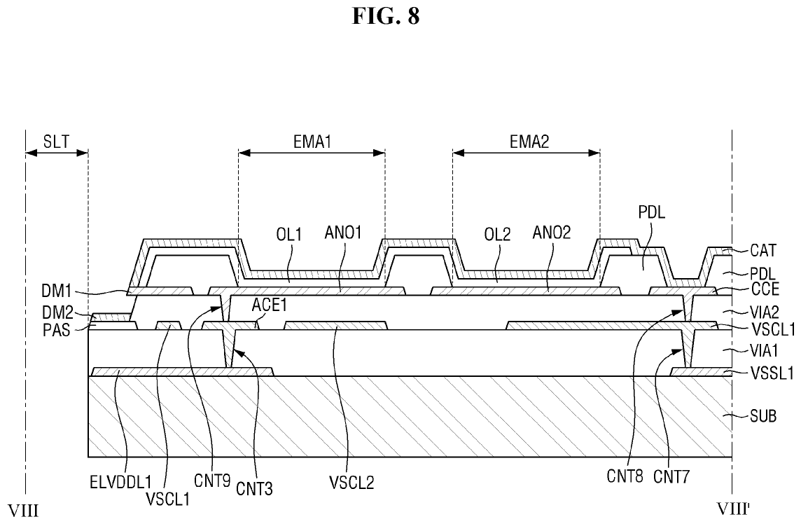

In an embodiment, the light therapy apparatus 10 may further include a first emission area EMA1, a second emission area EMA2, and a non-emission area NEM. As described later, the first emission area EMA1, the second emission area EMA2, and the non-emission area NEM may be divided by a bank layer PDL (see FIG. 8).

The first emission area EMA1 and the second emission area EMA2 may be disposed or defined in each array repeating unit APU. The first emission area EMA1 and the second emission area EMA2 may be disposed on each island ISL. The first emission area EMA1 may be an emission area of the first light emitting element LE1, and the second emission area EMA2 may be an emission area of the second light emitting element LE2. The first emission area EMA1 and the second emission area EMA2 may be divided by the non-emission area NEM.

The first emission area EMA1 and the second emission area EMA2 may have a same shape and a same size as each other in a plan view, but are not limited thereto. In an embodiment, for example, an area of the first emission area EMA1 in a plan view and an area of the second emission area EMA2 in a plan view may have a ratio of X:Y, where each of X and Y may be positive integer.

In an embodiment, as shown in FIG. 6, two emission areas EMA (the first emission area EMA1 and the second emission area EMA2) are disposed in each array repeating unit APU and each island ISL, but the number of emission areas EMA that may be disposed for each array repeating unit APU and each island ISL is not limited thereto, and may also be three or more.

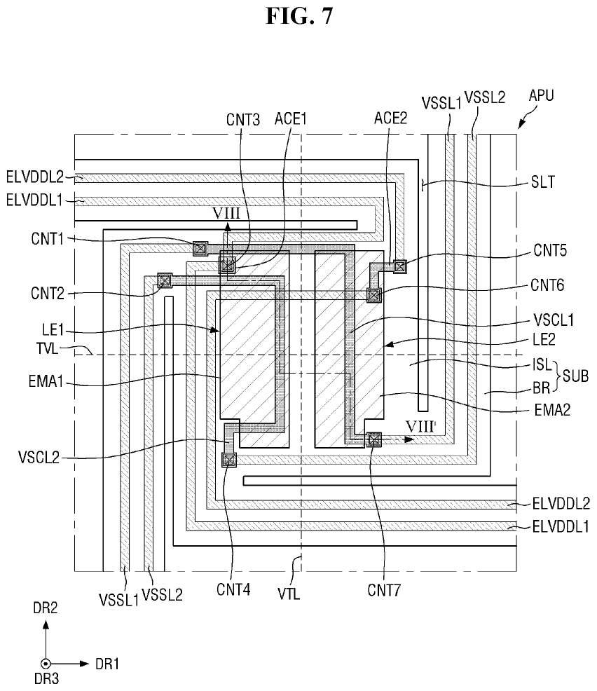

FIG. 7 is a plan view of an array repeating unit according to an embodiment. FIG. 8 is a cross-sectional view taken along line VIII-VIII′ of FIG. 7.

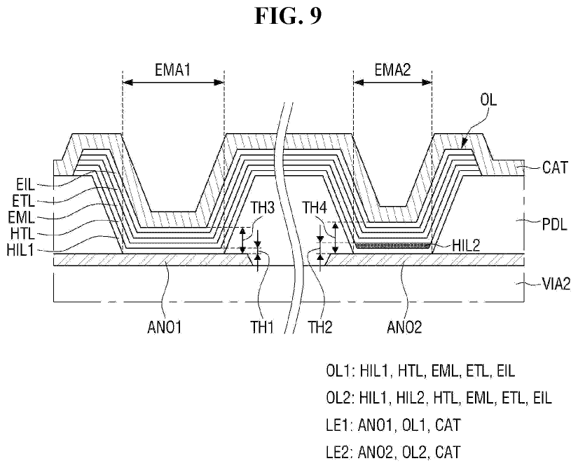

Referring to FIGS. 7 and 8, an embodiment of the light therapy apparatus 10 may further include a first conductive layer, a first planarization layer VIA1, a second conductive layer, a second planarization layer VIA2, a third conductive layer, a bank layer PDL, an organic layer OL (see FIG. 9), and a cathode electrode CAT.

The first conductive layer may be disposed on the substrate SUB. The first conductive layer may include first sub-source voltage lines ELVDDL1, second sub-source voltage lines ELVDDL2, third sub-source voltage lines VSSL1, and fourth sub-source voltage lines VSSL2. The first sub-source voltage lines ELVDDL1 and the second sub-source voltage lines ELVDDL2 may collectively define the first source voltage line ELVDDL shown in FIG. 4, and the third sub-source voltage lines VSSL1 and the fourth sub-source voltage lines VSSL2 may collectively define the second source voltage line ELVSSL shown in FIG. 4.

Each of the first sub-source voltage lines ELVDDL1 and the second sub-source voltage lines ELVDDL2 may be disposed over a plurality of islands ISL and a plurality of bridges BR neighboring to each other. The first sub-source voltage lines ELVDDL1 may be integrally formed with each other as a single unitary unit.

The second sub-source voltage lines ELVDDL2 may be connected to a second connection pattern ACE2 in an island ISL. In such an embodiment, the second sub-source voltage line ELVDDL2 disposed on the upper side of the island ISL and the second sub-source voltage line ELVDDL2 disposed on the lower side of the island ISL may be connected to each other by the second connection pattern ACE2. The second sub-source voltage line ELVDDL2 disposed on the upper side of the island ISL may be connected to the second connection pattern ACE2 through a fifth contact hole CNT5, and the second sub-source voltage line ELVDDL2 disposed on the lower side of the island ISL may be connected to the second connection pattern ACE2 through a sixth contact hole CNT6.

The third sub-source voltage lines VSSL1 and the fourth sub-source voltage lines VSSL2 may be disposed around the bridges BR, be separated from each other, and be provided in plural. The third sub-source voltage lines VSSL1 and the fourth sub-source voltage lines VSSL2 may be separated and electrically insulated from the first sub-source voltage lines ELVDDL1 and the second sub-source voltage lines ELVDDL2.

In an embodiment, the third sub-source voltage line VSSL1 disposed on the left side of the island ISL and the third sub-source voltage line VSSL1 disposed on the right side of the island ISL may be connected to each other by a first power connection pattern VSCL1. The third sub-source voltage line VSSL1 disposed on the left side of the island ISL may be connected to the first power connection pattern VSCL1 through a first contact hole CNT1, and the third sub-source voltage line VSSL1 disposed on the right side of the island ISL may be connected to the first power connection pattern VSCL1 through a seventh contact hole CNT7.

In an embodiment, the fourth sub-source voltage line VSSL2 disposed on the left side of the island ISL and the fourth sub-source voltage line VSSL2 disposed on the right side of the island ISL may be connected to each other by a second power connection pattern VSCL2. The fourth sub-source voltage line VSSL2 disposed on the left side of the island ISL may be connected to the second power connection pattern VSCL2 through a second contact hole CNT2, and the fourth sub-source voltage line VSSL2 disposed on the right side of the island ISL may be connected to the second power connection pattern VSCL2 through a fourth contact hole CNT4.

The first conductive layer may include at least one material selected from aluminum (Al), molybdenum (Mo), platinum (Pt), palladium (Pd), silver (Ag), magnesium (Mg), gold (Au), nickel (Ni), neodymium (Nd), iridium (Ir), chromium (Cr), calcium (Ca), titanium (Ti), tantalum (Ta), tungsten (W), and copper (Cu). The first conductive layer may be a single film or a multilayer film. In an embodiment, for example, the first conductive layer may be formed in a stacked structure of Ti/Al/Ti, Mo/Al/Mo, Mo/AlGe/Mo, Ti/Cu, or the like.

The first planarization layer VIA1 may be disposed on the first conductive layer. The first planarization layer VIA1 covers the first conductive layer. The first planarization layer VIA1 may be an interlayer insulating film or a via layer. In an embodiment where the first planarization layer VIA1 includes an organic material, an upper surface of the first planarization layer VIA1 may be substantially flat even in a case where a step structure is defined at a lower portion of the first planarization layer VIAL

The first planarization layer VIA1 may include an organic insulating material such as a polyacrylates resin, an epoxy resin, a phenolic resin, a polyamides resin, a polyimides resin, an unsaturated polyesters resin, a polyphenyleneethers resin, a polyphenylenesulfides resin, or benzocyclobutene (“BCB”).

The second conductive layer may be disposed on the first planarization layer VIAL. The second conductive layer may include the first power connection pattern VSCL1, the second power connection pattern VSCL2, a first connection pattern ACE1, and the second connection pattern ACE2. The first power connection pattern VSCL1, the second power connection pattern VSCL2, the first connection pattern ACE1, and the second connection pattern ACE2 may be disposed in each island ISL, and may be disposed in each array repeating unit APU.

The first connection pattern ACE1 may be electrically connected to the first sub-source voltage line ELVDDL1 to electrically connect the first sub-source voltage line ELVDDL1 to a first sub-electrode ANO1. The first connection pattern ACE1 may be in physical and/or electrical contact with the first sub-source voltage line ELVDDL1 through a third contact hole CNT3 defined through the first planarization layer VIA1 in the thickness direction to expose the first sub-source voltage line ELVDDL1.

The second connection pattern ACE2 may be electrically connected to the second sub-source voltage line ELVDDL2 to electrically connect the second sub-source voltage line ELVDDL2 to a second sub-electrode ANO2. The second connection pattern ACE2 may be in physical and/or electrical contact with the second sub-source voltage line ELVDDL2 through the fifth contact hole CNT5 and the sixth contact hole CNT6 defined through the first planarization layer VIA1 in the thickness direction to expose the second sub-source voltage line ELVDDL2.

The second conductive layer may include at least one material selected from the materials described above with respect to the first conductive layer. In an embodiment, the second conductive layer may include a same material as the first conductive layer and may have a same stacked structure as the first conductive layer, but is not limited thereto.

In an embodiment, the light therapy apparatus 10 may further include a passivation layer PAS. The passivation layer PAS may be disposed on the first planarization layer VIAL The passivation layer PAS may be disposed along an edge of the first planarization layer VIAL The passivation layer PAS may protrude toward the cutout portion SLT. A side surface of the passivation layer PAS may be disposed outside a side surface of the first planarization layer VIAL The side surface of the passivation layer PAS may protrude outward from the side surface of the first planarization layer VIAL Accordingly, the passivation layer PAS may have a tip shape (or an eaves shape) in the vicinity of an edge thereof. The passivation layer PAS may include an inorganic insulating material.

The second planarization layer VIA2 may be disposed on the second conductive layer and the passivation layer PAS. The second planarization layer VIA2 may cover the second conductive layer and cover a portion of the passivation layer PAS. The second planarization layer VIA2 may be an interlayer insulating film or a via layer. The second planarization layer VIA2 may include at least one organic insulating material selected from the materials list above with respect to the first planarization layer VIAL In an embodiment, the second planarization layer VIA2 may include a same material as the first planarization layer VIA1, but is not limited thereto.

The third conductive layer may be disposed on the second planarization layer VIA2. The third conductive layer may include a first electrode (or a first sub-electrode) ANO1 of the first light emitting element LE1, a first electrode (or a second sub-electrode) ANO2 of the second light emitting element LE2, a third connection pattern CCE, and a first dummy pattern DM1.

The first sub-electrode ANO1 may be in physical and/or electrical contact with the first connection pattern ACE1 through a ninth through hole CNT9 defined through the second planarization layer VIA2 in the thickness direction to expose the first connection pattern ACE1. The first sub-electrode ANO1 may be electrically connected to the first sub-source voltage line ELVDDL1 through the first connection pattern ACE1.

The second sub-electrode ANO2 may be in physical and/or electrical contact with the second connection pattern ACE2 through a through hole defined through the second planarization layer VIA2 in the thickness direction to expose the second connection pattern ACE2. The second sub-electrode ANO2 may be electrically connected to the second sub-source voltage line ELVDDL2 through the second connection pattern ACE2.

The third connection pattern CCE may not overlap the first sub-electrode ANO1 and the second sub-electrode ANO2. The third connection pattern CCE may be in physical and/or electrical contact with the first power connection pattern VSCL1 through an eighth through hole CNT8 defined through the second planarization layer VIA2 in the thickness direction to expose the first power connection pattern VSCL1. The cathode electrode CAT and the third sub-source voltage line VSSL1 may be electrically connected to each other through the third connection pattern CCE.

In an embodiment, the third connection pattern CCE may be in physical and/or electrical contact with the second power connection pattern VSCL2 through a through hole defined through the second planarization layer VIA2 in the thickness direction to expose the second power connection pattern VSCL2. The cathode electrode CAT and the fourth sub-source voltage line VSSL2 may be electrically connected to each other through the third connection pattern CCE.

The first dummy pattern DM1 may be disposed along an edge of the second planarization layer VIA2. The first dummy pattern DM1 may protrude toward the cutout portion SLT. A side surface of the first dummy pattern DM1 may be disposed outside a side surface of the second planarization layer VIA2. The side surface of the first dummy pattern DM1 may protrude outward from the side surface of the second planarization layer VIA2. Accordingly, the first dummy pattern DM1 may have a tip shape (or an eaves shape) in the vicinity of an edge thereof.

The third conductive layer may have a stacked film structure in which a material layer having a high work function and including indium tin oxide (“ITO”), indium zinc oxide (“IZO”), zinc oxide (ZnO), or indium oxide (In₂O₃) and a reflective material layer made of silver (Ag), magnesium (Mg), aluminum (Al), platinum (Pt), lead (Pd), gold (Au), nickel (Ni), neodymium (Nd), iridium (Ir), chromium (Cr), lithium (Li), calcium (Ca), or a combination thereof are stacked one on another, but is not limited thereto. The material layer having the high work function may be disposed on a layer above the reflective material layer to be disposed close to an organic layer OL (shown in FIG. 9). The third conductive layer may have a multilayer structure of ITO/Mg, ITO/MgF, ITO/Ag, and ITO/Ag/ITO, but is not limited thereto.

The bank layer PDL may be disposed on the third conductive layer. The bank layer PDL may at least partially overlap the non-emission area NEM. A plurality of openings, each exposing the first sub-electrode ANO1 or the second sub-electrode ANO2, may be defined through the bank layer PDL. The emission areas EMA1 and EMA2 and the non-emission areas NEM may be divided by the bank layer PDL. An area that does not overlap the bank layer PDL and overlaps the first sub-electrode ANO1 or the second sub-electrode ANO2 may become the emission area EMA1 or EMA2, and an area that overlaps the bank layer PDL may become the non-emission area EMA, but the disclosure is not limited thereto.

The bank layer PDL may include an inorganic insulating material such as silicon oxide, silicon nitride, silicon oxynitride, hafnium oxide, aluminum oxide, titanium oxide, tantalum oxide or zinc oxide, or an organic insulating material such as a polyacrylates resin, an epoxy resin, a phenolic resin, a polyamides resin, a polyimides resin, an unsaturated polyesters resin, a polyphenyleneethers resin, a polyphenylenesulfides resin or BCB. The bank layer PDL may be a single film or a multilayer film including stacked films including or made of different materials from each other.

An organic layer OL (see FIG. 9) is disposed in the openings of the bank layer PDL. The organic layer OL may be disposed on the first sub-electrode ANO1 and the second sub-electrode ANO2 exposed by the bank layer PDL. The organic layer OL may include an organic material. The organic layer OL may include an organic emission layer, a hole injection/transport layer, and an electron injection/transport layer. The organic layer OL may overlap the emission areas EMA1 and EMA2.

Hereinafter, the organic layer OL will be described in detail with reference to FIG. 9.

FIG. 9 is an enlarged view of the periphery of an organic layer of FIG. 8.

Referring to FIGS. 7 to 9, in an embodiment, the organic layer OL may include an organic layer (or a first intermediate layer) OL1 of the first light emitting element LE1 and an organic layer (or a second intermediate layer) OL2 of the second light emitting element LE2. The first intermediate layer OL1 may be positioned in an area where the first emission area EMA1 is disposed, and the second intermediate layer OL2 may be positioned in an area where the second emission area EMA2 is disposed. The first intermediate layer OL1 may be disposed between the first sub-electrode ANO1 and the cathode electrode CAT, and the second intermediate layer OL2 may be disposed between the second sub-electrode ANO2 and the cathode electrode CAT.

The first intermediate layer OL1 and the second intermediate layer OL2 may include a first hole injection layer HIL1, a hole transport layer HTL, an emission layer EML, an electron transport layer ETL, and an electron injection layer EIL. The first hole injection layer HILL the hole transport layer HTL, the emission layer EML, the electron transport layer ETL, and the electron injection layer EIL may be disposed over the first emission area EMA1 and the second emission area EMA2. In such an embodiment, the first light emitting element LE1 and the second light emitting element LE2 may share the first hole injection layer HILL the hole transport layer HTL, the emission layer EML, the electron transport layer ETL, and the electron injection layer EIL with each other. The second intermediate layer OL2 may further include a second hole injection layer HIL2.

Accordingly, a thickness of a hole injection layer of the first light emitting element LE1 may be substantially the same as a thickness (a width in the thickness direction) of the first hole injection layer HILL and a thickness of a hole injection layer of the second light emitting element LE2 may be substantially the same as the sum of a thickness (a width in the thickness direction) of the first hole injection layer HIL1 and a thickness (a width in the thickness direction) of the second hole injection layer HIL2. The thickness of the hole injection layer of the first light emitting element LE1 may be substantially the same as a distance between the first sub-electrode ANO1 and the hole transport layer HTL, and the thickness of the hole injection layer of the second light emitting element LE2 may be substantially the same as a distance between the second sub-electrode ANO2 and the hole transport layer HTL. The thickness of the hole injection layer of the first light emitting element LE1 may be a first thickness TH1, and the thickness of the hole injection layer of the second light emitting element LE2 may be a second thickness TH2. The second thickness TH2 may be greater than the first thickness TH1.

In an embodiment, a thickness of the organic layer (first intermediate layer) OL1 of the first light emitting element LE1 may be a third thickness TH3, and a thickness of the organic layer (second intermediate layer) OL2 of the second light emitting element LE2 may be a fourth thickness TH4. The fourth thickness TH4 may be greater than the third thickness TH3. The thickness of the organic layer (first intermediate layer) OL1 of the first light emitting element LE1 may be substantially the same as a distance between the first sub-electrode ANO1 and the cathode electrode CAT, and the thickness of the organic layer (second intermediate layer) OL2 of the second light emitting element LE2 may be substantially the same as a distance between the second sub-electrode ANO2 and the cathode electrode CAT.

The first hole injection layer HIL1 and the second hole injection layer HIL2 may serve to smoothly inject holes into the emission layer EML, and the hole transport layer HTL may serve to smoothly transport the holes. The electron injection layer EIL may serve to smoothly inject electrons into the emission layer EML, and the electron transport layer ETL may serve to smoothly transport the electrons. In the emission layer EML, the holes provided from the first hole injection layer HIL1 and/or the second hole injection layer HIL2 and the hole transport layer HTL and the electrons provided from the electron injection layer EIL and the electron transport layer ETL may react with each other to emit light.

The first light emitting element LE1 and the second light emitting element LE2 may emit light of different wavelengths from each other. In an embodiment, the first light emitting element LE1 includes the first hole injection layer HIL1 as the hole injection layer and the second light emitting element LE2 includes the first hole injection layer HIL1 and the second hole injection layer HIL2 as the hole injection layer, such that the thickness (first thickness TH1) of the hole injection layer of the first light emitting element LE1 and the thickness (second thickness TH2) of the hole injection layer of the second light emitting element LE2 may be different from each other, and the first light emitting element LE1 and the second light emitting element LE2 may emit the light of the different wavelengths from each other. In such an embodiment, the first light emitting element LE1 and the second light emitting element LE2 may emit light having different peak wavelengths from each other.

Light emitted from the light therapy apparatus 10 may have different therapeutic effects according to wavelengths thereof. In an embodiment, the light therapy apparatus 10 may independently emit light of wavelengths having different therapeutic effects, such that power consumption may be decreased. In an embodiment, where the light therapy apparatus 10 is portable, a driving time of the light therapy apparatus 10 may be increased, such that portability of the light therapy apparatus 10 may be improved.

In an embodiment, for example, a peak wavelength of the light emitted from the first light emitting element LE1 may be in a range of about 600 nanometers (nm) to about 630 nm, and a peak wavelength of the light emitted from the second light emitting element LE2 may be in a range of about 630 nm to about 660 nm, but the disclosure is not limited thereto.

In an embodiment, the second hole injection layer HIL2 is disposed only in the second emission area EMA2, and the first light emitting element LE1 and the second light emitting element LE2 share the first hole injection layer HILL the hole transport layer HTL, the emission layer EML, the electron transport layer ETL, and the electron injection layer EIL with each other, and thus, the number of mask processes is decreased, such that process efficiency may be improved and a process cost may be decreased.

Referring back to FIGS. 7 and 8, the cathode electrode CAT may be disposed on the organic layer OL. The cathode electrode CAT may be a second electrode of the light emitting element LE in itself or may include a second electrode of the light emitting element LE. The cathode electrode CAT may be disposed not only in the emission area EMA but also in the non-emission area NEM. The cathode electrode CAT may be disposed on and in direct contact with the first dummy pattern DM1 exposed without being covered by the bank layer PDL.

The cathode electrode CAT may include a material layer having a low work function, such as Li, Ca, LiF, Al, Mg, Ag, Pt, Pd, Ni, Au, Nd, Ir, Cr, BaF, Ba, or a combination (compound or mixture) thereof (e.g., a mixture of Ag and Mg, etc.), or materials with multilayer structures such as LiF/Ca or LiF/Al. The cathode electrode CAT may further include a transparent metal oxide layer disposed on the material layer having the low work function.

In an embodiment, the light therapy apparatus 10 may further include a second dummy pattern DM2. The second dummy pattern DM2 may include a same material as the cathode electrode CAT. The second dummy pattern DM2 may be disposed on and in direct contact with the passivation layer PAS exposed without being covered by the second planarization layer VIA2.

Hereinafter, a method for fabricating the light therapy apparatus according to an embodiment will be described.

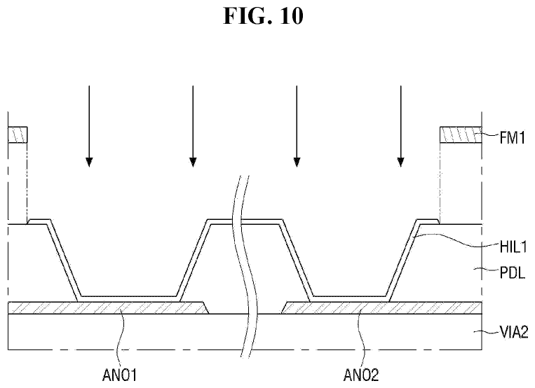

FIGS. 10 to 12 are cross-sectional views for describing a method for fabricating the light therapy apparatus according to an embodiment.

Referring to FIG. 10, in an embodiment of a method for fabricating the light therapy apparatus, the substrate SUB (see FIG. 8) is prepared, and the first conductive layer (see FIG. 8) disposed on the substrate SUB (see FIG. 8), the first planarization layer VIA1 (see FIG. 8), the second conductive layer (see FIG. 8), the second planarization layer VIA2, the third conductive layer (see FIG. 8) including the first sub-electrode ANO1 and the second sub-electrode ANO2, and the bank layer PDL with the openings exposing the first sub-electrode ANO1 and the second sub-electrode ANO2 are sequentially provided or formed on the substrate SUB.

The first hole injection layer HIL1 is formed on the first sub-electrode ANO1 and the second sub-electrode ANO2 through a first deposition mask FM1 exposing the openings exposing the first sub-electrode ANO1 and the second sub-electrode ANO2.

The first hole injection layer HIL1 may be formed over the opening exposing the first sub-electrode ANO1 and the opening exposing the second sub-electrode ANO2. The first hole injection layer HIL1 may be disposed on the first sub-electrode ANO1 exposed by the bank layer PDL and the second sub-electrode ANO2 exposed by the bank layer PDL.

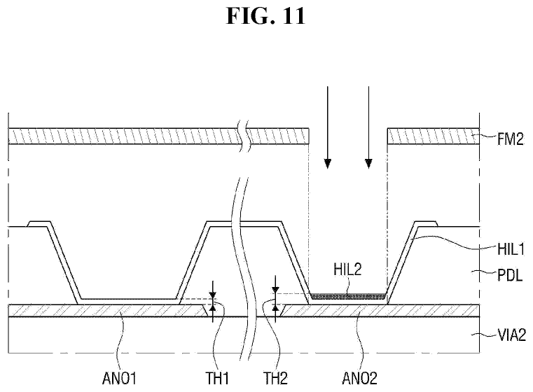

Next, referring to FIG. 11, the second hole injection layer HIL2 is formed on the second sub-electrode ANO2 through a second deposition mask FM2 exposing the opening exposing the second sub-electrode ANO2. The second deposition mask FM2 may cover the opening exposing the first sub-electrode ANO1 and expose the opening exposing the second sub-electrode ANO2. Accordingly, the second hole injection layer HIL2 may not be formed in the opening exposing the first sub-electrode ANO1, but may be formed in the opening exposing the second sub-electrode ANO2. The second hole injection layer HIL2 may be formed on the first hole injection layer HIL1 disposed on the second sub-electrode ANO2 exposed by the bank layer PDL.

The hole injection layer disposed on the first sub-electrode ANO1 and the hole injection layer disposed on the second sub-electrode ANO2 may be formed to have different thicknesses (first thickness TH1 and second thickness TH2), respectively, using two deposition masks by disposing the first hole injection layer HIL1 on the first sub-electrode ANO1 and the second sub-electrode ANO2 through the first deposition mask FM1 (see FIG. 10) and disposing the second hole injection layer HIL2 on the first hole injection layer HIL1 disposed on the second sub-electrode ANO2 through the second deposition mask FM2. Accordingly, the number of mask processes is decreased, such that process efficiency may be improved and a process cost may be decreased.

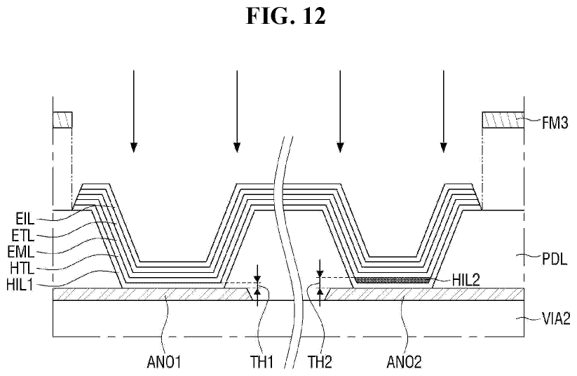

Next, referring to FIG. 12, the hole transport layer HTL, the emission layer EML, the electron transport layer ETL, and the electron injection layer EIL are formed through a third deposition mask FM3.

The third deposition mask FM3 may expose the openings exposing the first sub-electrode ANO1 and the second sub-electrode ANO2, and the hole transport layer HTL, the emission layer EML, the electron transport layer ETL, and the electron injection layer EIL may be formed over the opening exposing the first sub-electrode ANO1 and the opening exposing the second sub-electrode ANO2. In such an embodiment, the hole transport layer HTL, the emission layer EML, the electron transport layer ETL, and the electron injection layer EIL may be disposed on the first sub-electrode ANO1 exposed by the bank layer PDL and the second sub-electrode ANO2 exposed by the bank layer PDL.

The third deposition mask FM3 may be substantially the same as the first deposition mask FM1 (see FIG. 10), but is not limited thereto.

Hereinafter, alternative embodiments will be described. In such embodiments, any repetitive detailed description of same or like configurations as those of the embodiment described above will be omitted or simplified, and configurations different from those of the embodiment described above will be mainly described.

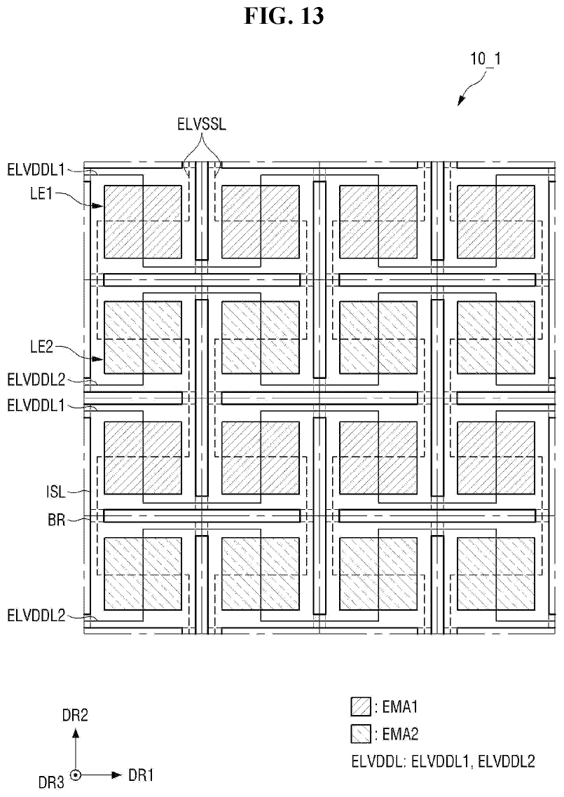

FIG. 13 is a plan view of a partial area of a light therapy apparatus according to an alternative embodiment.

A light therapy apparatus 10_1 shown in FIG. 13 is substantially the same as the light therapy apparatus 10 of FIG. 6 except that the first emission area EMA1 and the second emission area EMA2 are disposed on different islands ISL.

One of the first emission area EMA1 and the second emission area EMA2 may be disposed on each of a plurality of islands ISL, and one of a first light emitting element LE1 and a second light emitting element LE2 may be disposed on each of the plurality of islands ISL. The first emission areas EMA1 and the second emission areas EMA2 may be alternately and repeatedly disposed along the second direction DR2. The first emission areas EMA1 and the second emission areas EMA2 may be arranged along the first direction DR1, respectively. The first light emitting element LE1 may be positioned at a position where the first emission area EMA1 is disposed, and the second light emitting element LE2 may be positioned at a position where the second emission area EMA2 is disposed. In such an embodiment, the first light emitting element LE1 and the second light emitting element LE2 are substantially the same as those described above with reference to FIG. 8, and any repetitive detailed description thereof will be omitted.

The first sub-source voltage line ELVDDL1 of the first source voltage line ELVDDL may be electrically connected to the first light emitting element LE1, and the second sub-source voltage line ELVDDL2 of the first source voltage line ELVDDL may be electrically connected to the second light emitting element LE2. Each of the first sub-source voltage line ELVDDL1 and the second sub-source voltage line ELVDDL2 may extend substantially in the first direction DR1. The first sub-source voltage lines ELVDDL1 and the second sub-source voltage lines ELVDDL2 may be alternately and repeatedly disposed in the second direction DR2.

The second source voltage line ELVSSL may be electrically connected to the first light emitting element LE1 and the second light emitting element LE2. In a portion where the second source voltage line ELVSSL crosses the first sub-source voltage line ELVDDL1 or the second sub-source voltage line ELVDDL2, the second source voltage line ELVSSL may pass the first sub-source voltage line ELVDDL1 or the second sub-source voltage line ELVDDL2 through another conductive layer.

The first sub-source voltage line ELVDDL1 and the second sub-source voltage line ELVDDL2 may be electrically insulated from each other to independently apply signals. The first light emitting element LE1 and the second light emitting element LE2 may be independently driven. The first light emitting element LE1 and the second light emitting element LE2 may emit light of different wavelengths from each other, and at least one of the first light emitting element LE1 and the second light emitting element LE2 may be selected and driven based on a therapeutic purpose, such that power consumption of the light therapy apparatus 10_1 may be decreased.

In such an embodiment, a single emission area is disposed in each island ISL, and thus, the bank layer PDL (see FIG. 8) disposed between the emission areas may be omitted, such that an aperture ratio may be improved.

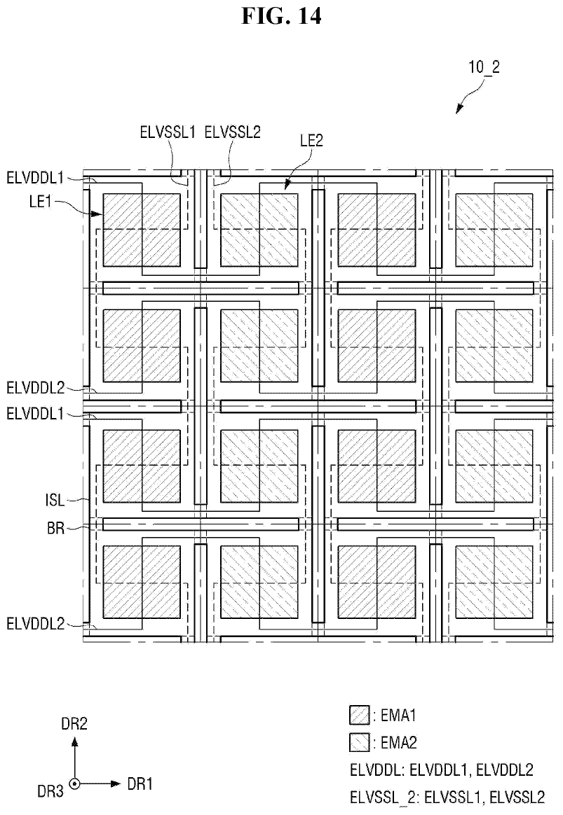

FIG. 14 is a plan view of a partial area of a light therapy apparatus according to another alternative embodiment.

A light therapy apparatus 10_2 shown in FIG. 14 is substantially the same as the light therapy apparatus 10_1 of FIG. 13 except that the first emission areas EMA1 and the second emission areas EMA2 are arranged along the second direction DR2, respectively.

In such an embodiment, the first emission areas EMA1 and the second emission areas EMA2 may be alternately and repeatedly disposed along the first direction DR1. The first emission areas EMA1 and the second emission areas EMA2 may be arranged along the second direction DR2, respectively. The first light emitting element LE1 may be positioned at a position where the first emission area EMA1 is disposed, and the second light emitting element LE2 may be positioned at a position where the second emission area EMA2 is disposed.

A second source voltage line ELVSSL_2 may include a third sub-source voltage line ELVSSL1 and a fourth sub-source voltage line ELVSSL2.

Each of the first sub-source voltage line ELVDDL1 and the second sub-source voltage line ELVDDL2 of the first source voltage line ELVDDL may extend substantially in the first direction DR1, and may be electrically connected to the first light emitting element LE1 and the second light emitting element LE2. The first sub-source voltage line ELVDDL1 and the second sub-source voltage line ELVDDL2 of the first source voltage line ELVDDL may be electrically insulated from each other, but are not limited thereto, and alternatively, the first sub-source voltage line ELVDDL1 and the second sub-source voltage line ELVDDL2 of the first source voltage line ELVDDL may be electrically connected to each other to apply substantially a same signal as each other.

The third sub-source voltage line ELVSSL1 of the second source voltage line ELVSSL_2 may be electrically connected to the first light emitting element LE1, and the fourth sub-source voltage line ELVSSL2 of the second source voltage line ELVSSL_2 may be electrically connected to the second light emitting element LE2. Each of the third sub-source voltage line ELVSSL1 and the fourth sub-source voltage line ELVSSL2 may extend substantially in the second direction DR2. The third sub-source voltage lines ELVSSL1 and the fourth sub-source voltage lines ELVSSL2 may be alternately and repeatedly disposed in the first direction DR1. The second source voltage line ELVSSL_2 may be electrically connected to the first light emitting element LE1 and the second light emitting element LE2.

The third sub-source voltage line ELVSSL1 may be electrically connected to the cathode electrode CAT (see FIG. 8) of the first light emitting element LE1, and the fourth sub-source voltage line ELVSSL2 may be electrically connected to the cathode electrode CAT (see FIG. 8) of the second light emitting element LE2. The light therapy apparatus 10_2 may have a step structure between the island ISL and the bridge BR in a cross-sectional view. In an embodiment, for example, the second planarization layer VIA2 (see FIG. 8) and the bank layer PDL (see FIG. 8) may be disposed on the island ISL, but the second planarization layer VIA2 (see FIG. 8) and the bank layer PDL (see FIG. 8) may not be disposed in at least a partial area on the bridge BR. However, the disclosure is not limited thereto.

In such an embodiment, due to the step structure, even though the cathode electrode CAT (see FIG. 8) is disposed over the entire area of the substrate SUB, the cathode electrode CAT (see FIG. 8) disposed on the island ISL and the cathode electrodes CAT (see FIG. 8) disposed on the bridge BR may be separated and electrically insulated from each other. In such an embodiment, the cathode electrodes CAT (see FIG. 8) disposed on the respective islands ISL may be separated and electrically insulated from each other.

Accordingly, the third sub-source voltage line ELVSSL1 and the fourth sub-source voltage line ELVSSL2 may be electrically insulated from each other to apply signals independently of each other. In such an embodiment, the first light emitting element LE1 and the second light emitting element LE2 may be driven independently of each other through the third sub-source voltage line ELVSSL1 and the fourth sub-source voltage line ELVSSL2. The first light emitting element LE1 and the second light emitting element LE2 may emit light of different wavelengths from each other, and at least one of the first light emitting element LE1 and the second light emitting element LE2 may be selected and driven based on a therapeutic purpose, such that power consumption of the light therapy apparatus 10_2 may be decreased.

In an embodiment, at least one of the first light emitting element LE1 and the second light emitting element LE2 may be selected and driven through the second source voltage line ELVSSL_2 as well as the first source voltage line ELVDDL, and thus, various designs may be possible as desired.

The invention should not be construed as being limited to the embodiments set forth herein. Rather, these embodiments are provided so that this disclosure will be thorough and complete and will fully convey the concept of the invention to those skilled in the art.

While the invention has been particularly shown and described with reference to embodiments thereof, it will be understood by those of ordinary skill in the art that various changes in form and details may be made therein without departing from the spirit or scope of the invention as defined by the following claims.

What is claimed is:

1. A light therapy apparatus comprising:
a first source voltage line including a first sub-source voltage line and a second sub-source voltage line, which are electrically insulated from each other;
a first light emitting element connected to the first sub-source voltage line, wherein the first light emitting element includes a first intermediate layer; and
a second light emitting element connected to the second sub-source voltage line, wherein the second light emitting element includes a second intermediate layer,
wherein
the first intermediate layer has a first thickness, and
the second intermediate layer has a second thickness greater than the first thickness.

2. The light therapy apparatus of claim 1, wherein
each of the first intermediate layer and the second intermediate layer include a first hole injection layer, a hole transport layer, an emission layer, an electron transport layer, and an electron injection layer, which are sequentially stacked one on another, and
the second intermediate layer further includes a second hole injection layer.

3. The light therapy apparatus of claim 2, wherein the second hole injection layer of the second intermediate layer is disposed between the first hole injection layer and the hole transport layer of the second intermediate layer.

4. The light therapy apparatus of claim 1, wherein a wavelength of light emitted from the first light emitting element and a wavelength of light emitted from the second light emitting element are different from each other.

5. The light therapy apparatus of claim 1, further comprising:
a substrate including a plurality of islands divided by a plurality of cutout portions and a plurality of bridges, each connecting adjacent islands among the plurality of islands to each other,
wherein the first source voltage line, the first light emitting element, and the second light emitting element are disposed on the substrate.

6. The light therapy apparatus of claim 5, wherein both of the first light emitting element and the second light emitting element are disposed on each of the plurality of islands.

7. The light therapy apparatus of claim 5, wherein one of the first light emitting element and the second light emitting element is disposed on each of the plurality of islands.

8. The light therapy apparatus of claim 7, wherein
the first light emitting elements are arranged along a first direction,
the second light emitting elements are arranged along the first direction, and
the first light emitting elements and the second light emitting elements are alternately disposed along a second direction crossing the first direction.

9. A light therapy apparatus comprising:
a substrate including a plurality of islands divided by a plurality of cutout portions and a plurality of bridges, each connecting adjacent islands among the plurality of islands to each other;
a first conductive layer disposed on the substrate, wherein the first conductive layer includes a first sub-source voltage line and a second sub-source voltage line, which are electrically insulated from each other;
a first planarization layer disposed on the first conductive layer;
a second conductive layer disposed on the first planarization layer, wherein the second conductive layer includes a plurality of first electrodes;
a bank layer disposed on the second conductive layer, wherein the bank layer exposes each of the plurality of first electrodes; and
an intermediate layer including a first intermediate layer disposed on a first sub-electrode of the plurality of first electrodes exposed by the bank layer and a second intermediate layer disposed on a second sub-electrode of the plurality of first electrodes exposed by the bank layer,
wherein
the first sub-source voltage line is electrically connected to the first sub-electrode, and
the second sub-source voltage line is electrically connected to the second sub-electrode.

10. The light therapy apparatus of claim 9, wherein
the first intermediate layer has a first thickness, and
the second intermediate layer has a second thickness greater than the first thickness.

11. The light therapy apparatus of claim 10, wherein
each of the first intermediate layer and the second intermediate layer include a first hole injection layer, a hole transport layer, an emission layer, an electron transport layer, and an electron injection layer that are sequentially stacked, and
the second intermediate layer further includes a second hole injection layer.

12. The light therapy apparatus of claim 11, wherein the second hole injection layer of the second intermediate layer is disposed between the first hole injection layer and the hole transport layer of the second intermediate layer.

13. The light therapy apparatus of claim 9, wherein one of the first sub-electrode and the second sub-electrode is disposed on each of the plurality of islands.

14. The light therapy apparatus of claim 13, wherein
the first sub-electrode and the second sub-electrode are arranged along a first direction, respectively, and
the first sub-electrode and the second sub-electrode are alternately disposed along a second direction crossing the first direction.

15. The light therapy apparatus of claim 14, wherein the first sub-electrode and the second sub-electrode are disposed on different islands among the plurality of islands, respectively.

16. The light therapy apparatus of claim 9, further comprising:
an emission area defined by the bank layer, wherein the emission area emits light,
wherein the emission area includes a first emission area overlapping the first sub-electrode and a second emission area overlapping the second sub-electrode.

17. The light therapy apparatus of claim 9, further comprising:
a cathode electrode disposed on the intermediate layer.

18. A method for fabricating a light therapy apparatus, the method comprising:
providing a first conductive layer including a first sub-electrode and a second sub-electrode on a substrate, providing a bank layer on the first conductive layer to expose each of the first sub-electrode and the second sub-electrode, and providing a first hole injection layer on the first sub-electrode and the second sub-electrode exposed by the bank layer; and
providing a second hole injection layer on the first hole injection layer disposed on the second sub-electrode,
wherein the providing the first hole injection layer is performed through a first deposition mask, and the providing the second hole injection layer is performed through a second deposition mask different from the first deposition mask,
the first deposition mask exposes the first sub-electrode and the second sub-electrode exposed by the bank layer, and
the second deposition mask covers the first sub-electrode exposed by the bank layer, and exposes the second sub-electrode exposed by the bank layer.

19. The method of claim 18, wherein
the first conductive layer, which is provided on the substrate, further includes a first sub-source voltage line and a second sub-source voltage line, which are electrically insulated from each other,
the first sub-source voltage line is electrically connected to the first sub-electrode, and
the second sub-source voltage line is electrically connected to the second sub-electrode.

20. The method of claim 18, wherein the substrate includes a plurality of islands divided by a plurality of cutout portions and a plurality of bridges, each connecting adjacent islands among the plurality of islands to each other.
